(19) United States
(12) Patent Application Publication
(10) Pub. No.: US 2017/0259016 A1
(43) Pub. Date: Sep. 14, 2017

(54) NEBULIZERS AND ASSOCIATED MEDICATION

(71) Applicant: Jynyvy Covarrubias, Valley Center, CA (US)

(72) Inventor: Jynyvy Covarrubias, Valley Center, CA (US)

(21) Appl. No.: 15/353,571

(22) Filed: Nov. 16, 2016

Related U.S. Application Data
(60) Provisional application No. 62/219,300, filed on Sep. 16, 2015.

(51) Int. Cl. A61M 11/02; A61K 36/324; A61K 31/05; A61K 36/185; A61K 9/00; A61K 47/10

(52) U.S. Cl. CPC A61M 11/02; A61K 9/0078; A61K 47/10; A61K 31/05; A61K 36/185; A61K 36/324; A61M 2205/025

(57) ABSTRACT

A novel nebulizer ruggedized to withstand high pressure and corrosive solutions that cannot be used in commercially available nebulizers. Also, a medication for use in such a nebulizer comprising cannabidiol (CBD) and an essential oil.

### FIELD OF THE INVENTION

The present invention relates to a handheld nebulizer and the use of such a device for administering medication to a patient, and the like.

### BACKGROUND

A jet nebulizer, also referred to as an atomizer, forces compressed gas to flow through a liquid, causing the liquid to aerosolize. This aerosol can then be inhaled by a patient. Relative to other technologies such as electronic cigarettes, where a heat source is used to vaporize a liquid, this process has little to no effect on the chemical composition of the material being atomized.

One common drawback of commercial nebulizers is that they use plastic or rubber components in their construction. As a result, they are not capable atomizing thick compounds such as heavy oils or glycerin based compounds. In addition to not being able to withstand the pressure required to nebulize these compounds over extended use, these non-durable components are also susceptible to corrosion if the liquid is in any way caustic.

As a result of these drawbacks, patients have been unable to reliably use nebulizers to inhale medicinal cannabis based medications that are suspended in glycerin or heavy oils. Furthermore, the mildly caustic nature of many essential oils and cannabis based medications means that they are also inappropriate for use in the nebulizers that are commercially available today.

### SUMMARY OF THE INVENTION

The above discussed disadvantages of the prior art are overcome by a nebulizer of the present invention that is fortified to withstand the pressures required to aerosolize heavy liquids such as heavy oils or glycerin.

Thus it is one aspect of some embodiments of the present invention to provide a nebulizer that lacks non-durable components, such as those made of plastic or rubber. In certain embodiments of the present invention, the components that comprise the nebulizer are made of a durable metal, such as stainless steel, or glass. In other embodiments, rubber gaskets may be replaced with Teflon equivalents.

It is another aspect of certain embodiments of the present invention to provide a medicinal cannabis based medication for use by a patient. In certain embodiments, such medication will comprise a substantially pure cannabidiol (CBD) extract suspended in a glycerin solution. In certain embodiments such a medication will comprise essential oils with medicinal properties, such as frankincense, in a glycerin solution.

Thus, one embodiment of the present invention provides, a method treating a patient with an aerosolized solution, the method comprising: i. placing the solution into the reservoir of a nebulizer; ii. forcing a gas into the solution; iii. inhaling the aerosolized solution; wherein the solution comprises at least one of a heavy oil and a glycerin based liquid, wherein the at least one of a heavy oil and a glycerin based liquid comprises a medication; wherein the nebulizer consists substantially of materials that are resistant to high pressure and corrosion; and wherein the nebulizer reservoir comprises an open end and a closed end, wherein said open end is removably attached to the aerosol generator, and said aerosol generator is attached to said reservoir such that said aerosol generator is nested within said reservoir;

Another embodiment of the present invention provides such a method, wherein said aerosol generator comprises a compressed gas receiver, a transfer tube, and an outlet tube, wherein said outlet tube is removably attached to said transfer tube.

Yet another embodiment of the present invention provides such a method, wherein said transfer tube is removably attached to said outlet tube.

Still another embodiment of the present invention provides such a method, wherein said nebulizer consists essentially of glass, teflon, and metal.

Yet another embodiment of the present invention provides such a method, wherein said metal comprises a nickel and rhodium coating.

Still another embodiment of the present invention provides such a method, wherein said medication comprises an extract from the cannabis genus of plants.

Yet another, wherein the extract comprises a substantially pure cannabidiol (CBD) in crystalline form.

Still another embodiment of the present invention provides such a method, wherein said extract comprises from 0.5 milligrams to 1000 milligrams of the substantially pure CBD per milliliter of liquid.

Yet another, wherein said essential oil comprises oil derived from at least one of frankincense, tea tree, peppermint, lavender, basil, bergamot, arborvitae, caramom, cinnamon bark, cilantro, clary sage, clove, coriander, eucalyptus, fennel, geranium, ginger, grapefruit, helichrysum, juniper berry, lemon, lemongrass, lime, marjoram, melaleuca, melissa, myrrh, patchouli, roman chamomile, rosemary, sandalwood, thyme, vetiver, wild orange, wintergreen, ylang ylang, angelica, cardamom, carrot seed, celery seed, cistus, copaiba, german chamomile, jasmine, lemon myrtle, melaleuca quiquenervia, orange, pine, rose, rose geranium, sage, spearmint, tangerine, tarragon, tea tree, xiang xao, and balsam fir.

Still another embodiment of the present invention provides such a method, wherein said essential oil comprises between 1 milligram and 1000 milligrams per milliliter of liquid.

Yet another embodiment of the present invention provides such a method, wherein said compressed gas is at least one of air or oxygen.

Still another embodiment of the present invention provides such a method, wherein said compressed gas is provided by at least one of a compressed air reservoir, a mechanical compressor, or a hand pump.

It is an alternate embodiment of the present invention to provide a kit for treating a patient, the kit comprising: i. a nebulizer, wherein the nebulizer consists of components capable of withstanding high pressure and corrosion; and ii. a medication, wherein the medication comprises at least one of a heavy oil and glycerin based solution, wherein the medication further comprises at least one of an extract from the cannabis genus of plants and an essential oil.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a line drawing of one embodiment of the nebulizer of the present invention.

FIG. 2 is a drawing of one embodiment of the reservoir of the nebulizer of the present invention.

FIG. 3 is a drawing of one embodiment of the aerosol generator of the present invention.

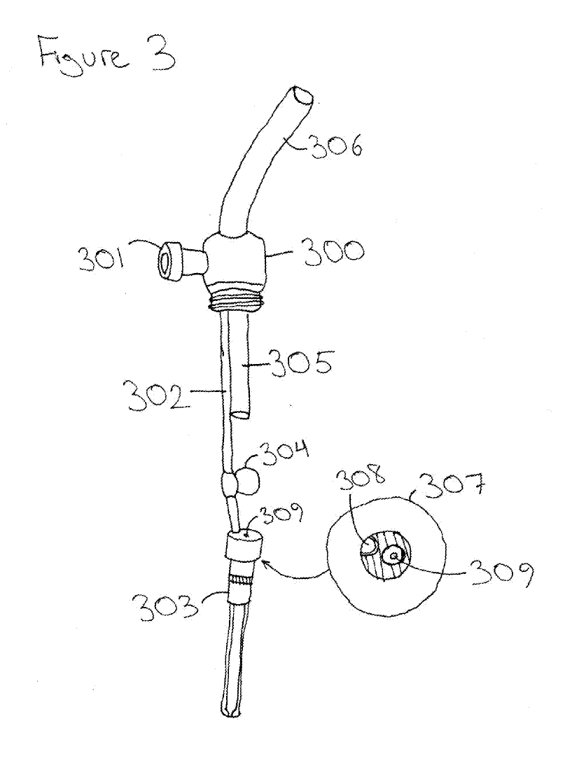

FIG. 4 is a drawing of one embodiment of the outlet tube of the present invention.

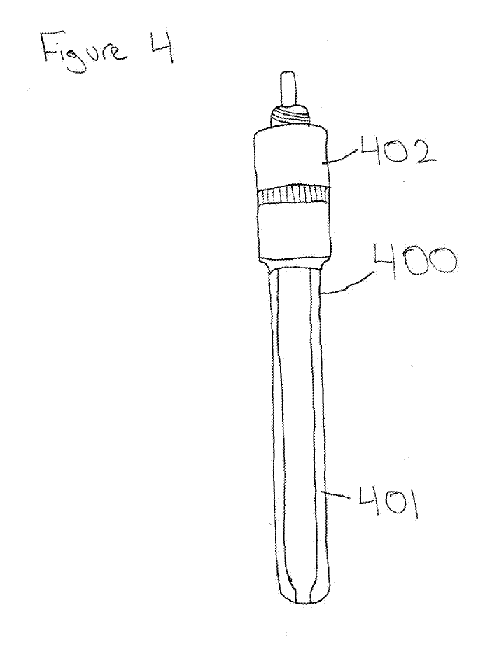

### DETAILED DESCRIPTION OF THE INVENTION

Unless otherwise defined, all terms used herein have the same meaning as commonly understood by one of ordinary skill in the art to which this invention belongs. Methods and materials are described herein for use of the present invention; other suitable methods and materials known in the art can also be used. The materials and methods, and examples are illustrative only and not intended to be limiting. All publications, patent applications, patents, and other references mentioned herein are incorporated by reference in their entirety. In case of conflict, the present specification including definitions will control.

Definitions:

“Aerosol generator” refers to any means of forcing a gas into a liquid to produce an aerosol. Although a preferred embodiment of a nebulizer is described herein, other configurations are possible. For example U.S. Pat. No. 5,054,477 to Terada, et. al. and U.S. Pat. No. 8,844,520 to Foley et. al. describe alternate nebulizer configurations.

“Medication” refers to any compound or composition which is used to treat at least one symptom of a patient.

“Patient” refers to any individual who is receiving or may receive a form of medical treatment. Patients include, but are not limited to, mammals such as humans, dogs, cats, and horses; birds; and other creatures which breath air.

“Substantially pure” refers to any compound which contains more of the referenced compound relative to all other impurities. Preferably, the composition will be at least 80%, and more preferably the composition will be 99% or more pure.

Referring now to the accompanying figures, it can be understood that the present invention relates to a ruggedized nebulizer and the use of such a device for administering a heavy liquid based medication to a patient.

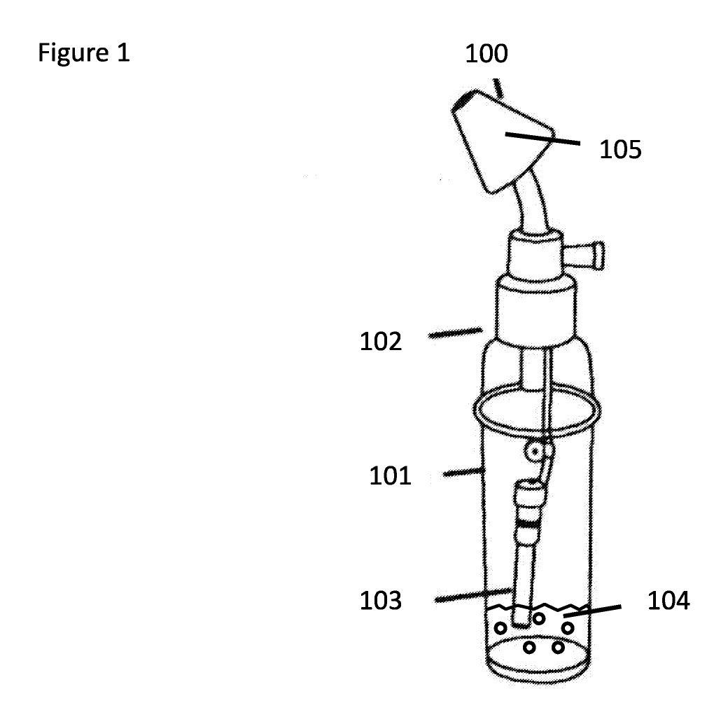

Turning now to FIG. 1, one embodiment of the nebulizer (100) of the present invention is presented. The nebulizer comprises a reservoir (101) that is removably attached to an aerosol generator (102). In the preferred embodiment, the aerosol generator (102) will nest within the reservoir (101) such that the outlet tube (103) of the aerosol generator is in contact with the medication (104) housed in the reservoir.

In a certain embodiments, the reservoir (200) will have an open end (201) and a closed end (202), and be substantially comprised of a material that is resistant to high pressures and corrosion. Preferably, the reservoir will comprise a durable clear material, such as glass. Though it is preferable that the reservoir be optically transparent, in order to enable one to monitor the amount of liquid (203) present in the reservoir, in certain embodiments the reservoir may be made of other materials capable of withstanding high pressures, such as various metals or ceramics.

In certain embodiments, the aerosol generator (300) comprises a compressed gas receiver (301), a transfer tube (302), and an outlet tube (303). Preferably, these components will be comprised of materials that are resistant to pressure and corrosion. More preferably, these components will comprise materials such as metal with a nickel and rhodium plating, and glass. In certain embodiments, the transfer tube may be equipped with a flow valve (304) to regulate the flow of gas being forced into the liquid contained in the reservoir.

In various embodiments, the compressed gas receiver (301) will be configured to attach to a hose. Such a hose could variably be attached to an air compressor, a tank of compressed gas, or a hand bulb. Alternately, such devices could be attached directly to the compressed gas receiver.

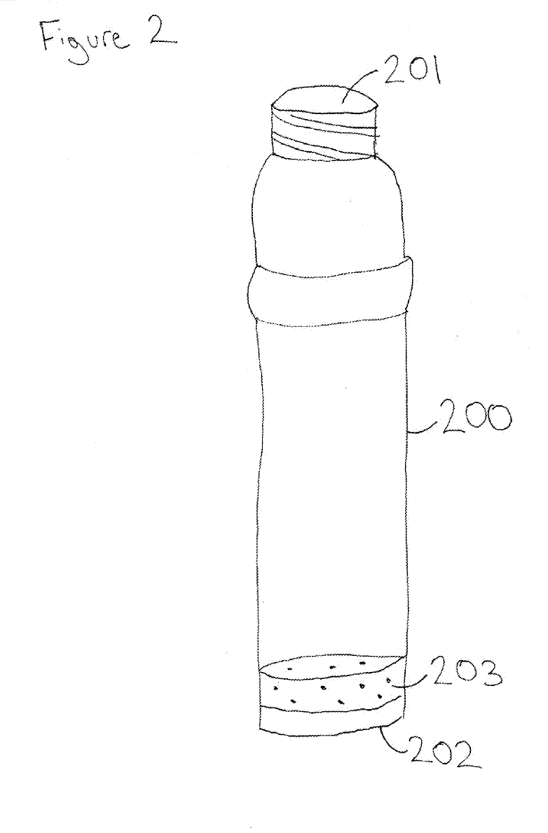

Also illustrated in FIG. 2 is an aerosol tube (205) and respiration outlet (306). The aerosol tube allows the atomized liquid from the nebulizer to be transferred from the reservoir to the patient. In certain embodiments, the aerosol tube may be incorporated into the aerosol generator, as shown. Alternately, the aerosol tube may be attached to a separate opening in the reservoir. The respiration outlet (306) may be attached to a component that facilitates respiration of the aerosol by the patient. In certain embodiments, this component may be a nasal respiration cone (105) either with or without vents, or a respiration mask. When viewed from the end with the outlet tube (303) removed (307), one can observe the end of the transfer tube (308), and the hole through which the aerosolized liquid enters the reservoir (309).

The outlet tube (400) may be comprised of a metal, glass, or both. Preferably, the outlet tube will be removably attached to the transfer tube, in order to facilitate cleaning of the components. More preferably, the outlet tube will comprise a glass outlet (401) attached to a metal connection (402), allowing the user to determine if the outlet tube requires cleaning.

In certain embodiments, the nebulizer of the present invention will be configured to force a gas, such as air or oxygen, into a liquid. This action will result in the liquid being particelized, and thus able to be easily inhaled by a patient.

It will be apparent to one having skill in the art that components resistant to high pressures or corrosive solutions could be retrofit onto existing nebulizer models. It is thus one embodiment of the present invention to provide such components in order to ruggedize an existing nebulizer.

Other embodiments of the present invention will provide a medication to be inhaled by the patient. This medication may comprise essential oils with medicinal properties, medicinal cannabis extracts, or other medications.

Cannabidiol (CBD) is a chemical that is commonly extracted from plants of the cannabis genus. One method of extracting CBD and a description of its properties is disclosed in US Patent Application No. 20060167283. Due to the presence of trace amounts of the acidic variant of cannabidiol (CBDA) present in most samples, even substantially pure CBD extract is mildly caustic, and thus unsuitable for use in traditional nebulizers. CBD has the following chemical structure:

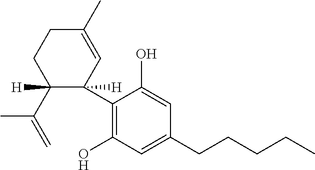 [SMILES: C=C(C)[C@@H]1CCC(C)=C[C@H]1c1c(O)cc(CCCCC)cc1O]

Treatment with CBD can provide numerous benefits. For example, 3 mg-10 mg of CBD can provide relief to Arthritis symptoms, inflammation, and pain; 25 mg-50 mg can provide relief of stress and anxiety symptoms; and 100 mg-200 mg per day can control many forms of epilepsy. As such, in certain embodiments of the present invention various concentrations of a substantially pure CBD extract are provided. These contractions range from 0.5 mg/mL, 1 mg/mL, 3 mg/mL, 10 mg/mL, 25 mg/mL, 50 mg/mL, 100 mg/mL, 200 mg/mL, 500 mg/mL, and 1000 mg/mL of substantially pure CBD to liquid base. Preferably, the liquid base will be oil or glycerin based. More preferably, the glycerin may be partially diluted with water. Most preferably, the glycerin to water ratio will be 8:7.

Cannabinol (CBN) is another compound that is present in may Cannabis species. Many beneficial effects have been attributed to this compound, including its use as a sedative, a pain relief agent, and an appetite stimulant. As such, certain embodiments of the present invention include various concentrations of a substantially pure CBN extract. These contractions range from 0.001 mg/mL, 0.5 mg/mL, 1 mg/mL, 3 mg/mL, 10 mg/mL, 25 mg/mL, 50 mg/mL, 100 mg/mL, 200 mg/mL, 500 mg/mL, and 1000 mg/mL of substantially pure CBN to liquid base. CBN has the following chemical structure:

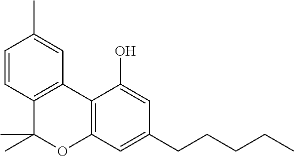 [SMILES: CCCCCc1cc(O)c2c(c1)OC(C)(C)c1ccc(C)cc1-2]

Tetrahydrocannabinol (THC) is yet another compound present in many Cannabis species. Many beneficial effects have been attributed to this compound, including its use as a an anti-anxiety agent, a pain relief agent, and an appetite stimulant. As such, certain embodiments of the present invention include various concentrations of a substantially pure THC extract. These contractions range from 0.001 mg/mL, 0.5 mg/mL, 1 mg/mL, 3 mg/mL, 10 mg/mL, 25 mg/mL, 50 mg/mL, 100 mg/mL, 200 mg/mL, 500 mg/mL, and 1000 mg/mL of substantially pure THC to liquid base. THC has the following chemical structure:

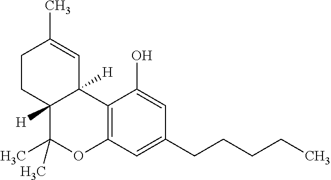 [SMILES: CCCCCc1cc(O)c2c(c1)OC(C)(C)[C@@H]1CCC(C)=C[C@@H]21]

Similarly, many essential oils are known to provide medicinal benefits. For example, and not as a limiting list, tea tree (Melaleuca alternifolia) oil has known antimicrobial properties, peppermint (Mentha piperita) oil has pain relieving, anti-inflammatory, and decongestant properties, lavender (Lavendula angustifolia) oil has analgesic properties, and frankincense (Boswellia frereana, Boswellia sacra and Boswellia carteri) oil has known anti-inflammatory and decongestant properties. Unfortunately, many of these oils are either caustic at the concentrations that would be useful in an inhaled medication, or are too thick to be used in a standard nebulizer.

Other embodiments of the present invention may use ingredients, including essential oils, derived from the following sources. Specific Latin names are provided by way of example, and are not meant to limit the disclosed source solely to the species listed, but rather to encompass all species which are known by that common name: Basil (Ocimum basilicum), Bergamot (Citrus bergamia), Arborvitae (Thuja plicata), Caramom (Ellettaria cardamomoum), Cinnamon Bark (Cinnamomum Zeylanicum), Cilantro (Coriandrum sativum), Clary Sage (Salvia sclarea), Clove (Eugenia caryophyllata), Coriander (Coriandrum sativum), Eucalyptus (Eucalyptus Radiata and Globias), Fennel (Foeniculum vulgare), Geranium (Pelargonium graveolens), Ginger (Zingiber officinale), Grapefruit (Citrus×paradisi), Helichrysum (Helichrysum italicum), Juniper Berry, Lemon (Citrus Limon), Lemongrass (Cymbopogon flexuosus), Lime (Citrus aurantifolia), Marjoram (Origanum majorana), Melaleuca (Melaleuca alternifolia), Melissa (Melissa officinalis), Myrrh (Commiphora myrrha), Patchouli (Pogostemon cablin), Peppermint (Mentha piperita), Roman Chamomile (Anthemis nobilis), Rosemary (Rosmarinus officinalis), Sandalwood (Santalum album and paniculatum), Thyme (Thymus vulgaris), Vetiver (Vetiveria zizanoides), Wild Orange (Citrus sinesis), Wintergreen (Gaultheria procumbens), Ylang Ylang (Canaga odorata), Angelica (Angelica archangelica), Cardamom (Elettaria cardamomum), Carrot Seed (Daucus carota), Celery Seed (Apium graveolens), Cistus (Cistus ladanifer), Copaiba, German Chamomile (Matricaria recutita), Jasmine (Jasaminum officianle), Lemon Myrtle, Melaleuca quiquenervia (Niaouli), Orange (Citrus sinsis), Pine (Pinus sylvestris), Rose (Rosa damascena), Rose Geranium, Sage (Salvia officinalis), Spearmint, Tangerine, Tarragon, Xiang Mao (Cymbopogon citratus), Balsam Fir, and Limonene.

In certain embodiments of the present invention, there is provided a medication containing a concentration of essential oil with medicinal properties. The concentration of essential oil in the medication may range from 1 mg/mL, 3 mg/mL, 5 mg/mL, 10 mg/mL, 50 mg/mL, 100 mg/mL, 500 mg/mL, 1000 mg/mL, and 5000 mg/mL of essential oil to liquid base. Preferably, the essential oil will be frankincense at a concentration of 500 mg/mL.

In a preferred embodiment, the medication will comprise 250 mg of substantially pure CBD crystilline and 500 mg of frankincense essential oil in a solution comprising 8 mL of glycerin and 7 mL of deionized water.

In another preferred embodiment, the medication will comprise 4.15 mg/mL of substantially pure THC, 0.12 mg/mL of substantially pure CBD, and 0.05 mg/mL of substantially pure CBN in a glycerin and water base.

In yet another embodiment of the present invention the medication will comprise 800 mg of substantially pure THC, 100-200 mg of substantially pure d-limonene, and 100-500 mg of frankincense suspended 15 mL of glycerin/water base.

Embodiments of the present invention may also contain various metallic particles to provide medicinal benefit. Such metals include, but are not limited to, gold, titanium, copper, and silver. Preferably, these metals will be suspended as a colloid in the solution to be administered to the patient. These particles may be provided in a concentration ranging from 0.01 micrograms per milliliter of solution to 1 milligram per milliliter of solution.

Methods and materials are described herein. However, methods and materials similar or equivalent to those described herein can be also used to obtain variations of the present invention. The materials, methods, and examples are illustrative only and not intended to be limiting.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A method treating a patient with an aerosolized solution, the method comprising:
i. placing the solution into the reservoir of a nebulizer;
ii. forcing a gas into the solution;
iii. inhaling the aerosolized solution;
wherein the solution comprises at least one of a heavy oil and a glycerin based liquid, wherein the at least one of a heavy oil and a glycerin based liquid comprises a medication;
wherein the nebulizer consists substantially of materials that are resistant to high pressure and corrosion; and
wherein the nebulizer reservoir comprises an open end and a closed end, wherein said open end is removably attached to the aerosol generator, and said aerosol generator is attached to said reservoir such that said aerosol generator is nested within said reservoir;

2. The method of claim 1, wherein said aerosol generator comprises a compressed gas receiver, a transfer tube, and an outlet tube, wherein said outlet tube is removably attached to said transfer tube.

3. The method of claim 2, wherein said transfer tube is removably attached to said outlet tube.

4. The method of claim 1, wherein said nebulizer consists essentially of glass, teflon, and metal.

5. The method of claim 4, wherein said metal comprises a nickel and rhodium coating.

6. The method of claim 1, wherein said medication comprises an extract from the cannabis genus of plants.

7. The method of claim 6, wherein the extract comprises a substantially pure cannabidiol (CBD) in crystalline form.

8. The method of claim 7, wherein said extract comprises from 0.5 milligrams to 1000 milligrams of the substantially pure CBD per milliliter of liquid.

9. The method of claim 1, wherein said medication comprises an essential oil, wherein said essential oil is derived from at least one of frankincense, tea tree, peppermint, lavender, basil, bergamot, arborvitae, caramom, cinnamon bark, cilantro, clary sage, clove, coriander, eucalyptus, fennel, geranium, ginger, grapefruit, helichrysum, juniper berry, lemon, lemongrass, lime, marjoram, melaleuca, melissa, myrrh, patchouli, roman chamomile, rosemary, sandalwood, thyme, vetiver, wild orange, wintergreen, ylang ylang, angelica, cardamom, carrot seed, celery seed, cistus, copaiba, german chamomile, jasmine, lemon myrtle, melaleuca quiquenervia, orange, pine, rose, rose geranium, sage, spearmint, tangerine, tarragon, tea tree, xiang xao, and balsam fir.

10. The method of claim 1, wherein said essential oil comprises between 1 milligram and 1000 milligrams per milliliter of liquid.

11. The method of claim 1 wherein said compressed gas is at least one of air or oxygen.

12. The method of claim 1, wherein said compressed gas is provided by at least one of a compressed air reservoir, a mechanical compressor, or a hand pump.

13. A kit for treating a patient, the kit comprising:
i. a nebulizer, wherein the nebulizer consists of components capable of withstanding high pressure and corrosion; and
ii. a medication, wherein the medication comprises at least one of a heavy oil and glycerin based solution, wherein the medication further comprises at least one of an extract from the cannabis genus of plants and an essential oil.
